(19) United States
(12) Patent Application Publication
(10) Pub. No.: US 2010/0036251 A1
(43) Pub. Date: Feb. 11, 2010

(54) DELAY ADJUSTMENT MODULE AND ULTRASONIC RECEIVING BEAM FORMING APPARATUS

(75) Inventor: Yoshitaka Baba, Tokyo (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/537,726

(22) Filed: Aug. 7, 2009

(30) Foreign Application Priority Data
Aug. 11, 2008 (JP) 2008-206834
Jun. 30, 2009 (JP) 2009-155824

(51) Int. Cl. A61B 8/14; G01V 13/00

(52) U.S. Cl. 600/447; 73/1.82

(57) ABSTRACT

A delay adjustment memory is shared by two signal processing channels, a signal in one signal processing channel having a smaller delay time of the two signal processing channels is adjusted in delay by the delay adjustment memory, and another signal in the other signal processing channel having a larger delay time is directly supplied to a subsequent calculation unit without passing through the delay adjustment memory, thereby performing the ultrasonic receiving beam forming processing.

### BACKGROUND OF THE INVENTION

1. Field of the Invention

The invention relates to an ultrasonic beam forming technique in an ultrasonic diagnosis apparatus.

2. Description of the Related Art

The conventional ultrasonic diagnosis apparatus adopts a method for transmitting ultrasonic waves to a subject to be inspected, especially in a living body, and receiving the returned reflective echo at a high accuracy by using an electrically scanning method.

An ultrasonic diagnosis apparatus transmits and receives ultrasonic beam through a probe with a plurality of micro transducers arranged one-dimensionally or two-dimensionally. In a transmission mode, a scan direction of the ultrasonic beam can be changed while changing a timing of applying a voltage to each micro transducer through each delay circuit. By changing delay time of each the delay circuits, the ultrasonic beam is scanned.

On the other hand, in a reception mode of the ultrasonic beam, reflective wave reflected from a target, point is received. However, the distance from a target point to each micro transducer is not constant. Therefore, the ultrasonic signals reflected from the target point arrive at the respective micro transducers at different times. Generally, in the ultrasonic receiving beam forming apparatus, a time lag (phase deviation) of the ultrasonic signals arriving at the different times is adjusted through phasing addition processing, and the ultrasonic beam is formed. In the phasing addition processing, an ultrasonic analog signal received by a micro transducer is amplified by an amplifier, analog-digital converted by an AD converter, and then the ultrasonic receiving digital signal is stored in a storing apparatus. The signal values coming from the same receiving surface are totaled at once in all necessary channels.

Further, in the ultrasonic receiving beam forming apparatus, the processing referred to as apodization is performed to improve the orientation of the one-dimensional or two-dimensional probe. This is the processing of not equally adding the echo signals received by the respective micro transducers within the probe but attenuating the echo signals positioned at the ends of a micro transducer array within the probe, to add them together. As a result, a force of the ultrasonic signal coming from a direction other than a target direction, which is called siderobe, can be restrained and the orientation of the micro transducer array can be improved. Generally, the respective echo signals received by the respective micro transducers are multiplied by the different weighting coefficients, to obtain the same effect as in the case of multiplication by a weighting function.

In the phasing addition processing of digital signals, a delay apparatus is used to adjust a delay time in every receiving channel. As the delay apparatus, a storing apparatus such as a First-In First-Out (FIFO) memory and a random access memory (RAM) is used.

The conventional ultrasonic diagnosis apparatuses aim to enhance their ability of diagnosis by obtaining much more ultrasonic receiving signals efficiently in the least number of times of transmitting and receiving ultrasonic wave, thereby improving the frame rate. Therefore, an ultrasonic receiving beam forming apparatus capable of forming multiple beams becomes necessary.

In the ultrasonic receiving beam forming apparatus capable of forming multiple beams, each different delay amount is adopted to every beam in every channel, and therefore, the configuration of the system becomes more complicated than in a case of obtaining one beam. Especially, an increase in the capacity of a memory used as the delay apparatus is remarkable. When the number of channels is 128, the maximum delay amount is 8000 clocks, and the data is 14 bits, the necessary memory capacity in the conventional ultrasonic receiving beam forming apparatus becomes 128×8000×14×1=14336000 b, about 14.4 Mb in a case of one beam. When the number of channels is 128, the maximum delay amount is 8000 clocks, and the data is 14 bits, the necessary memory capacity in the ultrasonic receiving beam forming apparatus capable of obtaining four beams becomes 128×8000×14×4=57344000 b, about 57.3 Mb.

In recent years, a high-speed readable/writable memory is installed in a field programmable gate array (FPGA) chip and FPGA chip have the ultrasonic receiving beam forming apparatuses installed. However, a high speed memory installed in the FPGA chip has a limit to the capacity and therefore, an ultrasonic receiving beam forming apparatus configurable with a small memory capacity is required. When the capacity of memory consumption in the ultrasonic receiving beam forming apparatus gets smaller, much more memory capacity can be used for another ultrasonic receiving signal processing circuit installed in the same FPGA chip. This improves the use efficiency of the FPGA chip and leads to the cost reduction of the system advantageously.

Japanese Patent Application Laid-Open No. 2002-336249 discloses a technique related to a receiving beam forming apparatus having delay elements in a multi-stage structure, which can process a plurality of scanning lines or beams with a smaller memory capacity than that of the conventional, ultrasonic receiving beam forming apparatus. In the former technique where a delay adjustment memory is arranged in every one channel, however, there are lots of wasteful memory regions not used. When receiving an ultrasonic signal from a direction of the maximum scanning angle, the largest amount of the delay adjustment memory is required in the receiving beam forming apparatus. Also in this case, there exist a lot of memory regions which are not used efficiently for the delay amount adjustment.

### SUMMARY OF THE INVENTION

An object of the present invention is to provide an ultrasonic receiving beam forming apparatus capable of forming beam with a small amount of memory capacity while reducing the memory capacity which is not actually effectively used for delay amount adjustment in the ultrasonic signal receiving.

A delay adjustment module according to the invention is a delay adjustment module for receiving two ultrasonic receiving signals and adjusting a time lag between the signals, including:

a storing unit for absorbing the time lag between the signals, and

a circuit connecting unit which switches a connection of the respective signals to a subsequent-stage circuit by comparing delay time of the two signals, wherein

the storing unit is shared by two signal processing channels, and

the circuit connecting unit switches a connection so that one signal having a smaller delay of the two signals is output through the storing unit and the other signal having a larger delay is output directly.

Further, a delay adjustment module according to the invention is a delay adjustment module for receiving N (N is integer of 3 or more) ultrasonic receiving signals and adjusting a time lag among the signals, including:

a storing unit for absorbing the time lag among the signals, and

a circuit connecting unit which switches a connection of the respective signals to a subsequent-stage circuit by comparing delay time of the signals, wherein

there are N−1 pieces of the storing units, and

the circuit connecting unit switches a connection depending on the delay time of each of the signals so that the signal is output through the storing unit corresponding to the delay time thereof or output directly.

An ultrasonic receiving beam forming apparatus according to the invention including:

the delay adjustment module, and

an adding unit, which adds ultrasonic receiving signals after the time lag between the signals is adjusted by the delay adjustment module.

According to the invention, it is possible to form an ultrasonic receiving beam forming apparatus with a small amount of memory capacity while reducing the memory capacity which is not actually effectively used for the delay amount adjustment in the ultrasonic signal receiving.

Further features of the present invention will become apparent from the following description of exemplary embodiments with reference to the attached drawings.

### BRIEF DESCRIPTION OF THE DRAWINGS

FIG. 1 shows an example of the configuration of an ultrasonic receiving beam forming apparatus according to a first embodiment.

FIG. 2 shows an example of the configuration of the ultrasonic receiving beam forming apparatus according to the first embodiment.

FIG. 3 shows an example of the configuration of the ultrasonic receiving beam forming apparatus according to the first embodiment.

FIG. 4 shows an example of the configuration of an ultrasonic receiving beam forming apparatus according to a second embodiment.

FIG. 5 shows an example of the configuration of the ultrasonic receiving beam forming apparatus according to the second embodiment.

FIG. 6 shows an example of the configuration of the ultrasonic receiving beam forming apparatus according to the second embodiment.

FIG. 7 shows an example of the configuration of an ultrasonic receiving beam forming apparatus according to a third embodiment.

FIG. 8 shows an example of the configuration of the ultrasonic receiving beam forming apparatus according to the third embodiment.

FIG. 9 shows an example of the process of signal processing in the ultrasonic diagnosis apparatus.

FIG. 10 shows an example of the configuration of an ultrasonic image creating system according to a fourth embodiment.

FIG. 11 shows an example of the configuration of a delay adjustment memory control circuit according to the fourth embodiment.

FIG. 12 shows an example of the configuration of a weighting coefficient supplying circuit.

FIG. 13 shows an example of the connected state of the units according to the fourth and fifth embodiments.

FIG. 14 shows an example of the connected state of the units according to the fourth embodiment.

FIG. 15 shows an example of the configuration of a delay adjustment memory control circuit according to the fifth embodiment.

FIG. 16 shows an example of the configuration of the delay adjustment memory control circuit according to the fifth embodiment.

FIG. 17 shows an example of the connected state of the units according to the fifth embodiment.

FIG. 18 shows an example of the connected state of the units according to the fifth embodiment.

FIG. 19 shows an example of the configuration of an NA-NB order comparing circuit according to the fifth embodiment.

### DESCRIPTION OF THE EMBODIMENTS

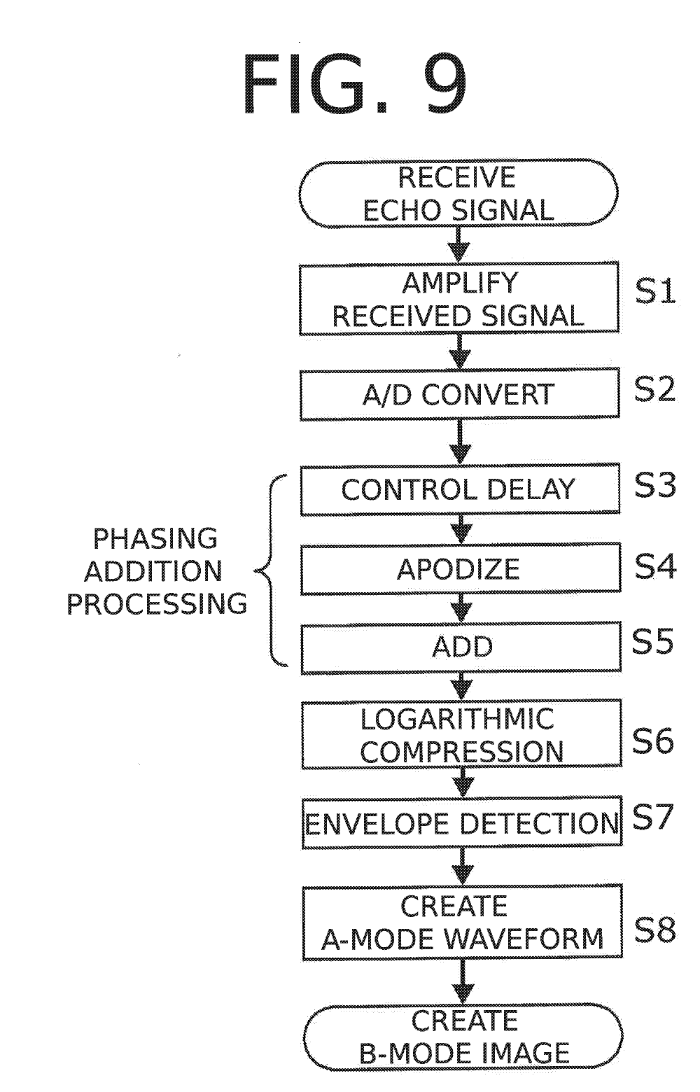

FIG. 9 is a flow chart of an example of the process of signal processing for obtaining a B mode image in an ultrasonic diagnosis apparatus. The received analog echo signal is amplified (S1) by a Low Noise Amplifier and a Variable Gain Amplifier, and then converted into digital by analog digital conversion (S2). Thereafter, it is subjected to the phasing addition processing (delay control (S3), apodization (S4), addition (S5)), the logarithmic compression (S6), and the envelope detection (S7), hence to create an A mode waveform (S8) and further to build a B mode image. The ultrasonic receiving beam forming apparatus according to the invention is used for the phasing addition processing (S3 to S5) shown in FIG. 9. It is needless to say that the ultrasonic receiving beam forming apparatus according to the invention can be used in the phasing addition processing in a flow of signal processing other than shown in FIG. 9.

Hereinafter, embodiments according to the invention will be described in detail with reference to the drawings.

### First Embodiment

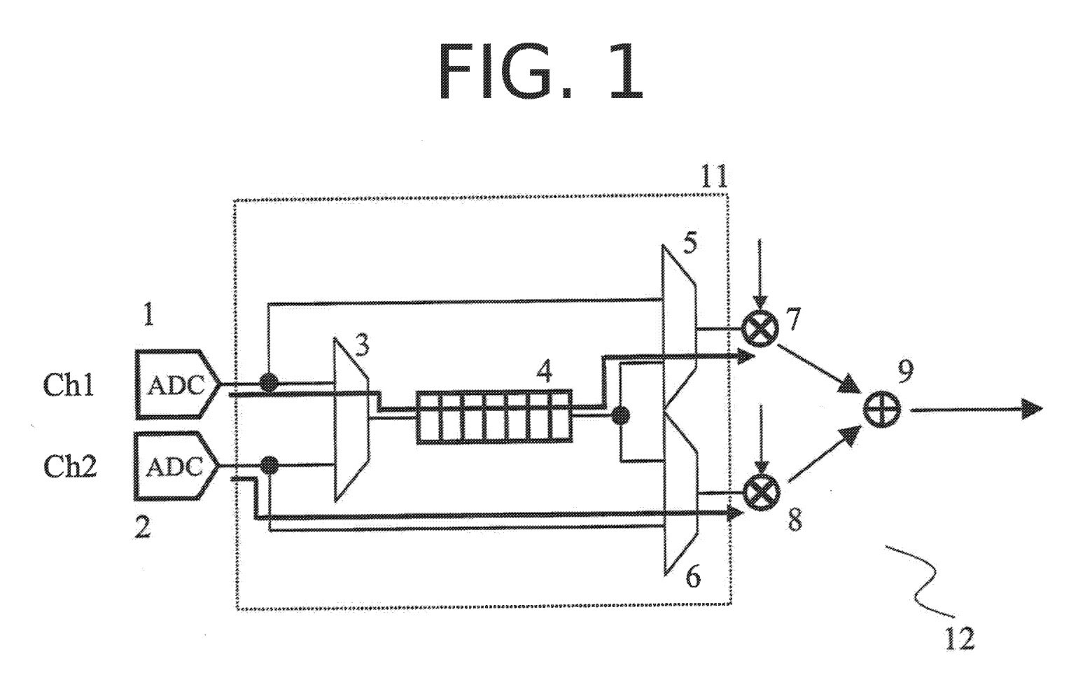

FIG. 1 shows the configuration of an ultrasonic receiving beam forming apparatus for two channels according to a first embodiment of the invention.

This ultrasonic receiving beam forming apparatus 12 includes an AD converter 1 connected to the channel 1 and an AD converter 2 connected to the channel 2. Further, it includes a delay adjustment module 11 for adjusting a delay time between the both channels. It further includes multipliers 7 and 8 for performing the apodization processing on the delay adjusted signal to improve the orientation and an adder 9 for adding the signals of the both channels.

The delay adjustment module 11 includes a multiplexer 3 which connects either the output of the AD converter 1 or the output of the AD converter 2 to a delay adjustment memory 4, by comparison of delay time between the channel 1 and the channel 2. Here, the delay time means a time taken for the transmitted ultrasonic wave to arrive at a target point, being reflected there, and to arrive at the ultrasonic receiving element.

Further, it includes a multiplexer 5 which connects either the output of the AD converter 1 or the output of the delay adjustment memory 4 to the multiplier 7 for the channel 1, by comparison of the delay time between the channel 1 and the channel 2. It further includes a multiplexer 6 which connects either the output of the AD converter 2 or the output of the delay adjustment memory 4 to the multiplier 8 for the channel 2, by comparison of the delay time between the channel 1 and the channel 2. It includes an adder 9 which adds the output results of the multiplier 7 and the multiplier 8. The delay adjustment memory may be formed by using a FIFO memory (First-In First-Out memory) or a random access memory. The delay adjustment memory 4 corresponds to the storing unit of the invention and the multiplexers 3, 5, and 6 correspond to the circuit connecting unit of the invention.

The ultrasonic receiving data received in the respective channels are supplied to the AD converters 1 and 2 and sampled there. The sampled data from the AD converters 1 and 2 should be adjusted in the delay time, to obtain beam from a desired direction. Here, the multiplexers 3, 5, and 6 receive a signal of the comparison result as the result of comparing the delay time of the sampled data received from the channel 1 with that from the channel 2. According to the comparison result, a channel having a smaller delay of the channels 1 and 2 is connected to the delay adjustment memory 4 and the other channel having a larger delay is directly connected to the multipliers 7 and 8. A delay time is supplied from, a delay time storing memory, that is a peripheral circuit of the ultrasonic receiving beam forming apparatus, or a delay time calculation circuit (not illustrated) and the comparison of the delay time is performed by a comparator (comparing circuit). The comparator (comparing circuit) compares the size of the delay time data given to the channel 1 with that of the channel 2 and supplies a selection signal including the connection information to the multiplexer. According to this, such a signal that has a smaller delay time of the two signals and first arrives at the receiving element is passed to the delay adjustment memory 4, thereby making it possible to adjust a time lag between signals.

More specifically, each distance from a target point to each ultrasonic receiving element is various and each period of time (delay time) taken for each ultrasonic receiving signal reflected from a target point to arrive at the respective ultrasonic receiving elements or the respective ultrasonic receiving channels, is various. Therefore, the ultrasonic receiving beam forming apparatus detects an ultrasonic receiving signal reflected from a target point while adjusting each delay time of the signals received from the respective ultrasonic receiving elements. The delay time is adjusted also on the respective channels of the ultrasonic receiving beam forming apparatus shown in FIG. 1, to obtain an ultrasonic receiving signal reflected from a target point. Here, in the invention, a comparison of the delay time is performed between the adjacent two channels. The data in a channel having a smaller delay of the two channels or a channel positioned nearer to a target point is supplied to the delay adjustment memory 4 and further supplied to a circuit in the subsequent-stage through the delay adjustment memory 4. The delay adjustment memory 4 needs to have a capacity enough to store the ultrasonic receiving digital data only for the delay difference between the two channels to enable this delay adjustment. While, the data in the other channel having a larger delay or the other channel positioned at a distance from the target point is directly supplied to a circuit in the subsequent-stage.

The circuit operation will be described, for example, in a case where the channel 1 is nearer to the target point, than the channel 2, or where the delay time of the channel 1 is shorter than that of the channel 2. Here, the output, of the AD converter 1 for the channel 1 is connected to the delay adjustment memory 4, and the output of the delay adjustment memory 4 is connected to the multiplier 7 for the channel 1, through the multiplexers 3 and 5. On the other hand, the output of the AD converter 2 for the channel 2 is directly connected, to the multiplier 8 for the channel 2 through the multiplexers 3 and 6. In this situation of the connection, the ultrasonic receiving digital data arriving at the channel 1 is accumulated in the delay adjustment memory 4. The ultrasonic receiving signal arriving at the channel 2 is directly supplied to the multiplier 8, where a weighting coefficient for apodization is attached to the signal. The receiving digital data in the channel 1 is read out from the delay adjustment memory 4 so that the multiplier 7 may attach a weighting coefficient for apodization to the receiving signal received in the channel 1 at the same timing as the above. The ultrasonic receiving signals with the delay time adjusted and the weighting coefficients for apodization attached by the multipliers 7 and 8 are added together in the adder 9.

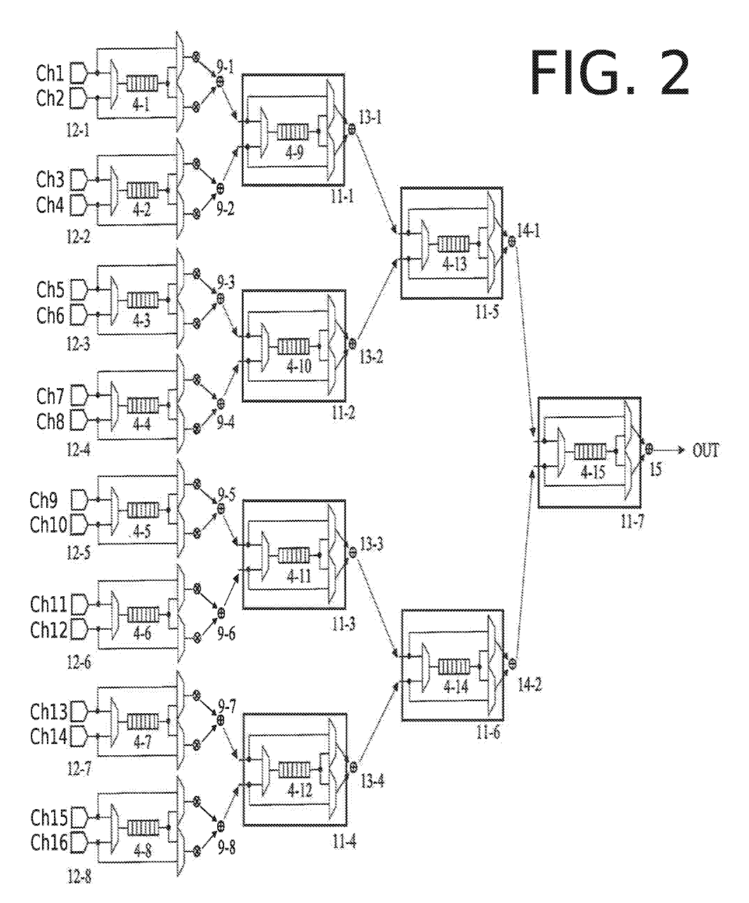

FIG. 2 shows the configuration of the ultrasonic receiving beam forming apparatus in which the circuit of FIG. 1 is used in a case where the number of channels is more than two. As illustrated in FIG. 2, the circuits of FIG. 1 are formed in multi stages, and the signals in all the channels are phased and added. The multipliers for apodization processing are provided only in the first stage and no multiplier is used in the second stage and the later.

The delay adjustment modules 11-1 to 11-7 connected in the next stage after the adders 9-1 to 9-8 should be provided with delay adjustment memories each having a capacity enough to adjust a delay time between the adjacent signal processing channels. Namely, each of the delay adjustment memories 4-9 to 4-15 of the respective delay adjustment modules 11-1 to 11-7 needs to have a capacity enough to store the ultrasonic receiving digital data for the maximum delay difference (the ultrasonic receiving digital data corresponding to a maximum difference of delay time) between the adjacent signal processing channels. According to this structure, it is possible to adjust the delay time for all the channels finally.

For example, the delay adjustment modules 11-1 to 11-4 positioned in the next stage after the adders 9-1 to 9-8 should adjust each delay time difference of the signals added and supplied by the delay adjustment modules 12-1 to 12-8 positioned in the first stage. Each of the delay adjustment memories 4-9 to 4-12 needs to have a memory capacity enough to store the ultrasonic digital data for a delay difference between the two channels. The delay adjustment modules 11-5 and 11-6 positioned in the further next stage after the adders 13-1 to 13-4 should adjust each delay time difference between the signals added and supplied by the delay adjustment modules 11-1 to 11-4. Then, each of the delay adjustment memories 4-13 and 4-14 needs to have a memory capacity enough to adjust the delay time for four channels.

According to this structure, the capacity of the delay adjustment memory used in the ultrasonic receiving beam forming apparatus can be reduced much more than in the conventional example. In a case of the ultrasonic receiving beam forming apparatus having, for example, 128 channels, this embodiment enables the phasing addition processing with some percent of the capacity of the delay adjustment memory used in the conventional ultrasonic receiving beam forming apparatus.

A necessary memory capacity will be calculated in the first embodiment of the invention. Assume that the number of channels is 126, the maximum delay amount is 8000 clocks, data is 14 bits, and one beam is obtained with equal delay time between the channels. In this case, the memory capacity of 64×(8000/128)×14=56448 b, about 56.4 Kb is required in every one adding stage. According to the first embodiment of the invention, six adding stages are generated in a case of the 128 channels and a total of the necessary memory capacity becomes 56448 b×6=338688 b, about 339 Kb. This is about 2.4% of the memory capacity in the conventional example.

Further, by operating the ultrasonic receiving beam forming apparatus at the clock frequency several times more than the sampling frequency in the ultrasonic diagnosis apparatus, a plurality of beams can be obtained. For example, in the case of that the sampling frequency of the ultrasonic diagnosis apparatus is 40 MHz, when the ultrasonic receiving beam forming apparatus can be operated at 160 MHz, four times more than the above, four beams can be obtained through one transmission and reception, thereby improving the frame rate.

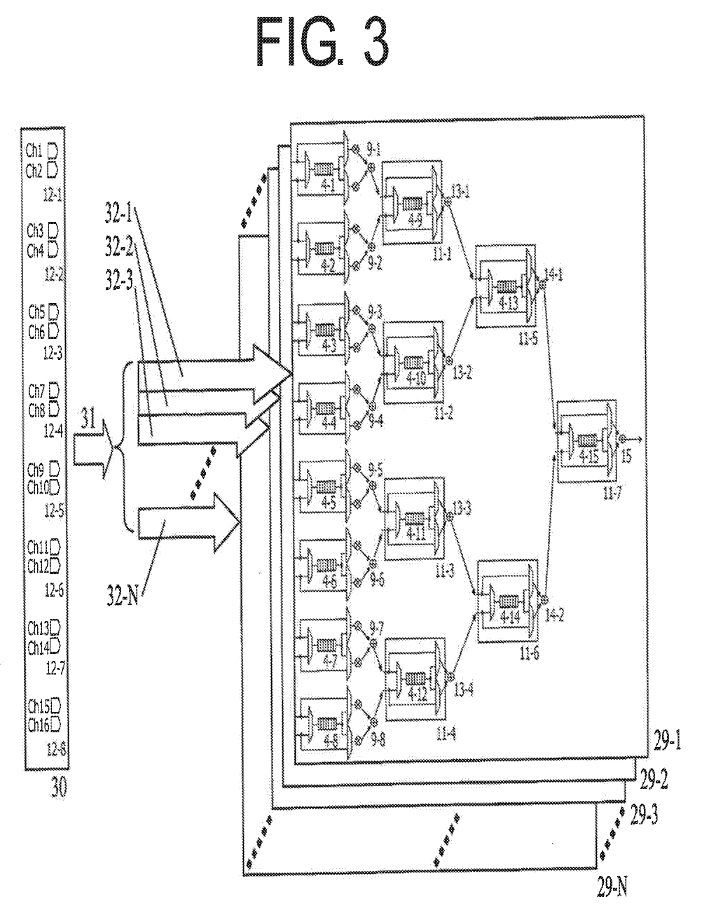

The ultrasonic receiving beam forming apparatus, however, has a limit to the operation frequency. When a user wants to obtain the number of beams (multiple beams) more than the number of (the maximum operation frequency/sampling frequency in the ultrasonic receiving beam forming apparatus), a plurality of the ultrasonic receiving beam forming apparatuses may be installed in parallel, as illustrated in FIG. 3 (29-1 to 29-N). In this case, it is not necessary to arrange the AD converters in parallel but a plurality of ultrasonic receiving beam forming apparatuses 29-1 to 29-N may share an AD converter group 30 and a plurality of the structures 29 subsequent to the AD converters of the ultrasonic receiving beam forming apparatuses may be arranged in parallel. The output 31 from the AD converter group 30 may be distributed to the respective ultrasonic receiving beam forming apparatuses 29-1 to 29-N through distribution channels 32-1 to 32-N.

According to the first embodiment of the invention, since the capacity of the delay adjustment memory in the ultrasonic receiving beam forming apparatus is small, the number of the ultrasonic receiving beam forming apparatuses that can be installed in parallel, within the FPGA chip can be increased. When it is compared with the conventional example, much more beams can be obtained with the same memory capacity. Also in the conventional example, the number of the beams that can be obtained from one ultrasonic receiving beam forming apparatus has the upper limit of the value of the maximum operation frequency/sampling frequency of the ultrasonic receiving beam forming apparatus. A memory capacity usable as the delay adjustment memory is restricted; therefore, according as the delay adjustment memory capacity gets smaller in one ultrasonic receiving beam forming apparatus, the number of the ultrasonic receiving beam forming apparatuses that could be arranged in parallel can be increased. This results in increasing the number of the beams that can be obtained.

### Second Embodiment

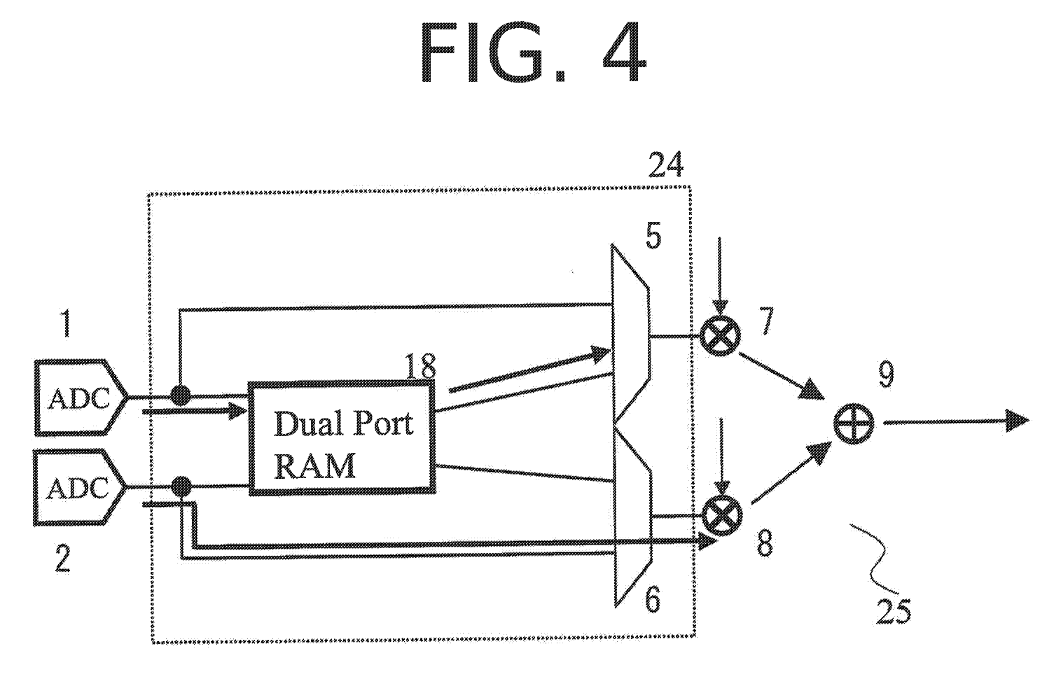

FIG. 4 shows a second embodiment of the invention. In the first embodiment, a FIFO memory or a RAM of single port is used as the delay adjustment memory 4; however, in this embodiment, a circuit is formed using a RAM of dual port.

In the ultrasonic diagnosis apparatus, the ultrasonic receiving beam forming apparatus is installed using the Field programmable Gate Array (FPGA) in many cases. The recent FPGA chip often has a high-speed writable and readable RAM mounted there and the mounted RAM can be used as a dual port memory. In this case, the delay adjustment memory 4 and the multiplexer 3 in FIG. 1 are replaced with a dual port memory 18 mounted in the FPGA chip), thereby realizing the same operation as that of the circuit shown in FIG. 1.

The ultrasonic receiving data received in the respective channels are supplied to the AD converters 1 and 2 and sampled there. The sampled data from the AD converters 1 and 2 should be adjusted in the delay time to obtain beam from a desired direction. Here, the dual port memory 18 and the multiplexers 5 and 6 receive a signal of the comparison result as the result of comparing the delay time of the sampled data received from the channel 1 with that from the channel 2. According to the comparison result, the data in a channel having a smaller delay is supplied to the dual port memory 18, while the data in the other channel having a larger delay is directly supplied to the multipliers 7 and 8. A delay time is supplied from a delay time storing memory, that is a peripheral circuit of the ultrasonic receiving beam forming apparatus, or a delay time calculation circuit, (not illustrated) and the comparison of the delay time is performed by a comparator (comparing circuit). The comparator (comparing circuit) compares the size of the delay time data given to the channel 1 with that of the data given to the channel 2 and supplies a selection signal including the connection information to the multiplexer. According to this, such a signal that has a smaller delay time and first arrives at the receiving element is passed to the dual port memory 18, thereby making it possible to adjust a time lag between signals.

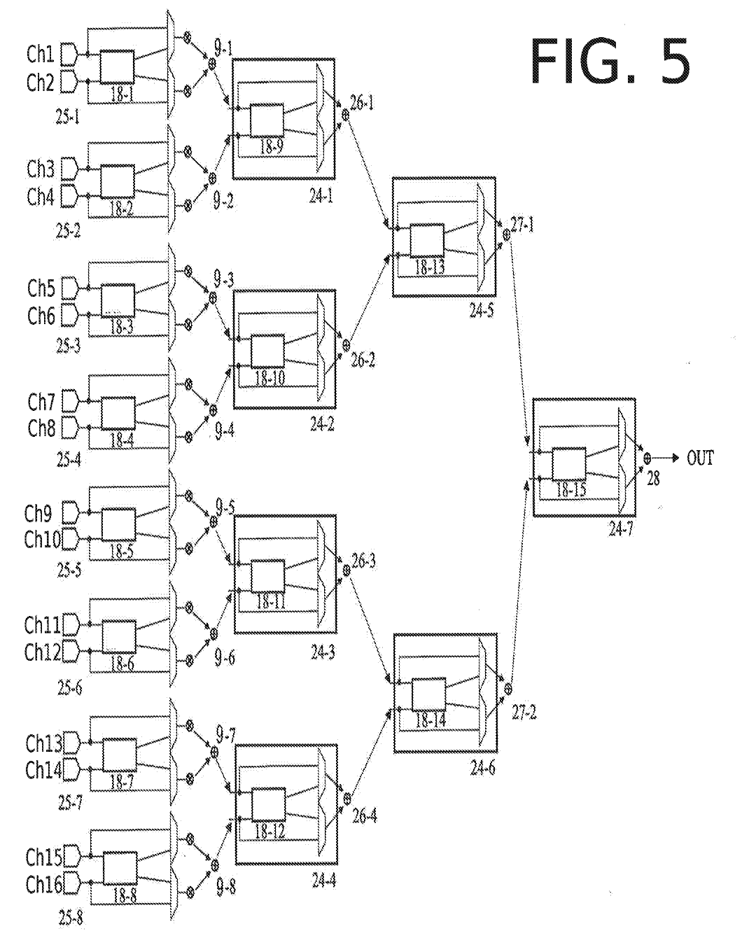

FIG. 5 shows the configuration of the ultrasonic receiving beam forming apparatus in which the circuit of FIG. 4 is used in a case where the number of channels is more than two. The delay adjustment modules 24-1 to 24-7 connected in the next stage after the adders 9-1 to 9-8 should be provided with delay adjustment memories each having a capacity enough to adjust a delay time between the adjacent ultrasonic receiving beam forming apparatuses. According to this structure, it is possible to adjust the delay time for all the channels finally.

For example, the delay adjustment modules 24-1 to 24-4 positioned in the next stage after the adders 9-1 to 9-8 should adjust each delay time difference of the signals added and supplied by the delay adjustment modules 25-1 to 25-8 positioned in the first stage. Each of the delay adjustment memories 18-9 to 18-12 needs to have a memory capacity capable of adjusting the delay time for the two channels. The delay adjustment modules 24-5 to 24-6 positioned in the further next stage after to the adders 26-1 to 26-4 should adjust each delay time difference between the signals added and supplied by the delay adjustment modules 24-1 to 24-4. Then, each of the delay adjustment memories 18-13 and 18-14 needs to have a memory capacity capable of adjusting the delay time for four channels.

According to this structure, the capacity of the delay adjustment memory used in the ultrasonic receiving beam forming apparatus can be reduced much more than in the conventional example. A necessary memory capacity will be calculated in the second embodiment, of the invention. Assume that the number of channels is 128, the maximum delay amount is 8000 clocks, data is 14 bits, and one beam is obtained with equal delay time between the channels. In this case, the memory capacity of 64×(8000/128)×14 b=56448 b, about 56.4 Kb is required in every one adding stage. According to the second embodiment of the invention, six adding stages are generated in a case of the 128 channels and a total of the necessary memory capacity becomes 56448 b×6=338688 b, about 339 Kb. This is about 2.4% of the memory capacity in the conventional example.

Further, by operating the ultrasonic receiving beam forming apparatus at the clock frequency several times more than the sampling frequency in the ultrasonic diagnosis apparatus, a plurality of beams can be obtained. In the case of that the sampling frequency of the ultrasonic diagnosis apparatus is, for example, 40 MHz, when the ultrasonic receiving beam forming apparatus can be operated at 160 MHz, four times more than the above, four beams can be obtained through one transmission and reception, thereby improving the frame rate.

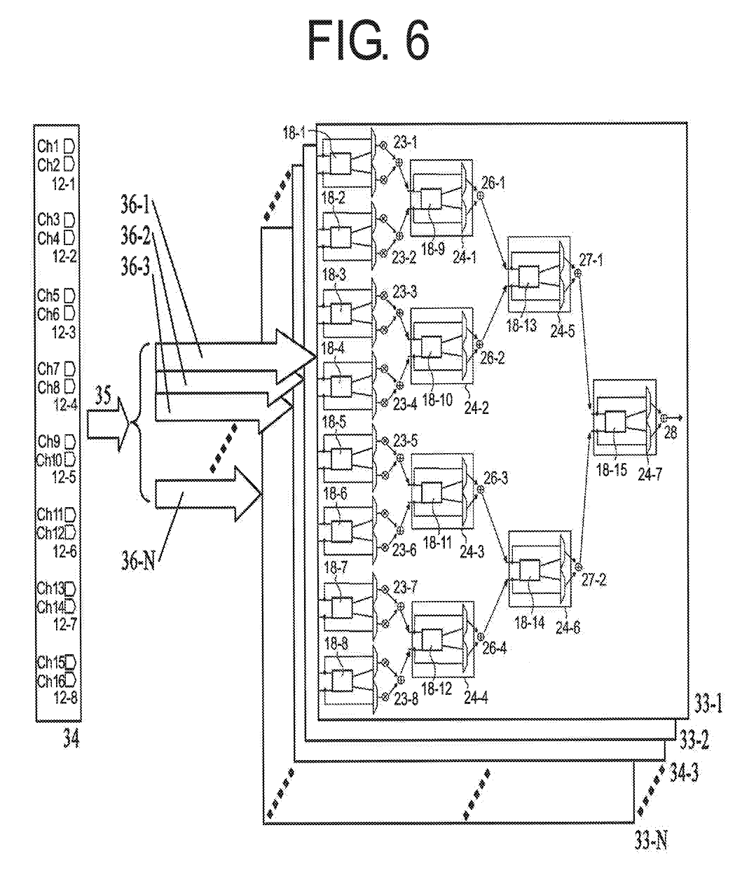

The ultrasonic receiving beam forming apparatus, however, has a limit to the operation frequency. When a user wants to obtain the number of beams more than the number of the maximum operation frequency/sampling frequency in the ultrasonic receiving beam forming apparatus, a plurality of the ultrasonic receiving beam forming apparatuses may be installed in parallel, as illustrated in FIG. 6 (33-1 to 33-N). In this case, it is not necessary to arrange the AD converters in parallel but a plurality of ultrasonic receiving beam forming apparatuses 33-1 to 33-N may share an AD converter group 34 and a plurality of the structures 33 subsequent to the AD converters of the ultrasonic receiving beam, forming apparatuses may be arranged in parallel. The output 35 from the AD converter group 34 may be distributed to the respective ultrasonic receiving beam forming apparatuses 33-1 to 33-N through distribution channels 36-1 to 36-N.

According to the second embodiment of the invention, the capacity of the delay adjustment memory in the ultrasonic receiving beam forming apparatus can be decreased. Therefore, similarly to the first embodiment, when it is compared with the conventional example, much more beams can be obtained with the same memory capacity.

### Third Embodiment

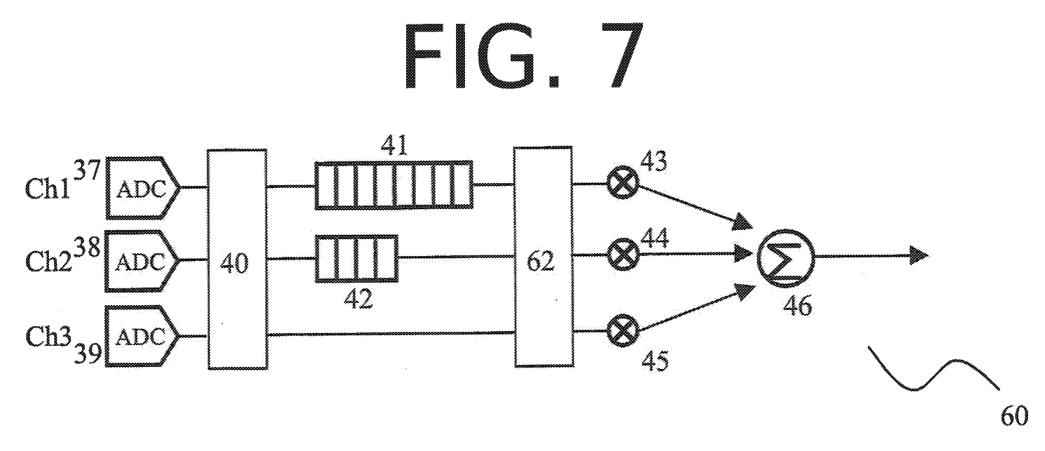

FIG. 7 shows a third embodiment, of the invention. Although the first and second embodiments perform the ultrasonic beam forming by comparison of the delay time between the two channels, the number of channels is not limited to two. FIG. 7 shows an example of using three channels, in which two delay adjustment memories are used. A delay adjustment memory 41 has a capacity capable of adjusting the delay time for two channels and a delay adjustment memory 42 has a capacity capable of adjusting the delay time for one channel. In this embodiment, delay time is compared among three channels by a comparator (comparing circuit), a channel having the smallest delay is connected to the delay adjustment memory 41, and another channel having the second smallest delay is connected to the delay adjustment memory 42. The channel having the largest delay is connected not to the delay adjustment memory but directly to a subsequent-stage circuit. According to this structure, it is possible to adjust, the delay time for all the channels finally. The connection is controlled by switching circuits 40 and 62 according to the output from the comparator (comparing circuit).

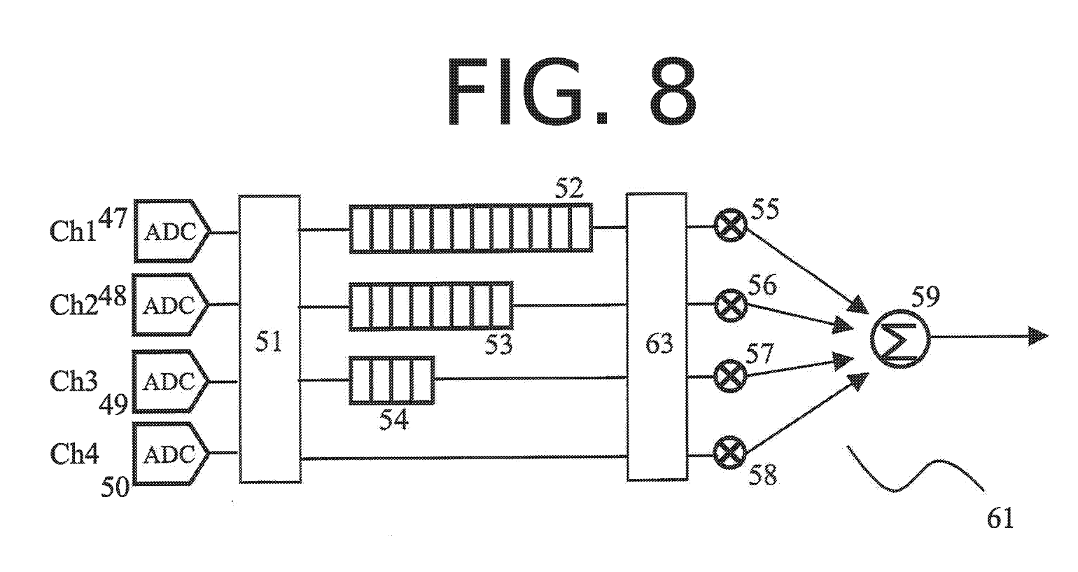

Alternatively, as illustrated in FIG. 8, four channels may be used. Generally speaking, when the number of channels is N (N is the integer of 3 or more), (N−1) pieces of delay adjustment memories are used and the connection of the respective signals to the subsequent-stage circuits is switched according to each delay time of the signals in the respective channels. The N−1 delay adjustment memories respectively have a capacity capable of adjusting the delay time for the maximum delay difference among two to N signals. The switching circuit connects a signal having the smallest delay to the delay adjustment memory having the maximum capacity and connects a signal having the second smallest delay to the delay adjustment memory having the second largest capacity, and outputs a signal having the largest delay directly to the subsequent-stage circuit.

A necessary memory capacity will be calculated in a case of adopting the structure shown in FIG. 8 to this embodiment. Assume that the number of channels is 128, the maximum delay amount is 8000 clocks, data, is 14 bits, and one beam is obtained with equal delay time among the channels. In this case, in every four channels, three delay adjustment memories, that are a delay adjustment memory 52 for three channels, a delay adjustment memory 53 for two channels, arid a delay adjustment memory 54 for one channel, are arranged. Therefore, the first adding stage requires a memory capacity of (3+2+1)×128/4×(8000/128)×14 b=168000 b, 168 Kb. The second adding stage requires a memory of (12+8+4)×(8000/128)×14 b=21000 b, 21 Kb, in every four signal channels in a case of adopting the configuration of adjusting delay collectively in the four signal channels. Therefore, the second adding stage requires a memory capacity of a total of 21 Kb×128/4/4=168 Kb. The third adding stage requires a memory capacity of (48+32+16)×(8000/128)×14 b=84000 b, 84 Kb, in every four signal channels in a case of the configuration of adjusting delay collectively in the four signal channels. Therefore, the third adding stage requires a memory capacity of a total of 84 Kb×128/4/4/4=168 Kb. In the final adding stage, delay adjustment is performed on two signal channels and it requires a memory of 128/2×(8000/128)×14 b=56000 b, 56 Kb. According to the third embodiment of the invention, in a case of 128 channels, the total sum of the required memory capacity becomes 168 Kb+168 Kb+168 Kb+56 Kb=560 Kb. This is about 3.9% of the memory capacity of 14.4 Mb in the conventional example.

Further, it is needless to say that similarly to the first and second embodiments, a plurality of beams can be obtained also in a case of the ultrasonic receiving beam, forming apparatus according to the third embodiment of the invention. Also in this embodiment, the ultrasonic receiving beam forming apparatus may be operated at the clock frequency several times more than the sampling frequency and a plurality of the ultrasonic receiving beam forming apparatuses may be installed in parallel.

### Fourth Embodiment

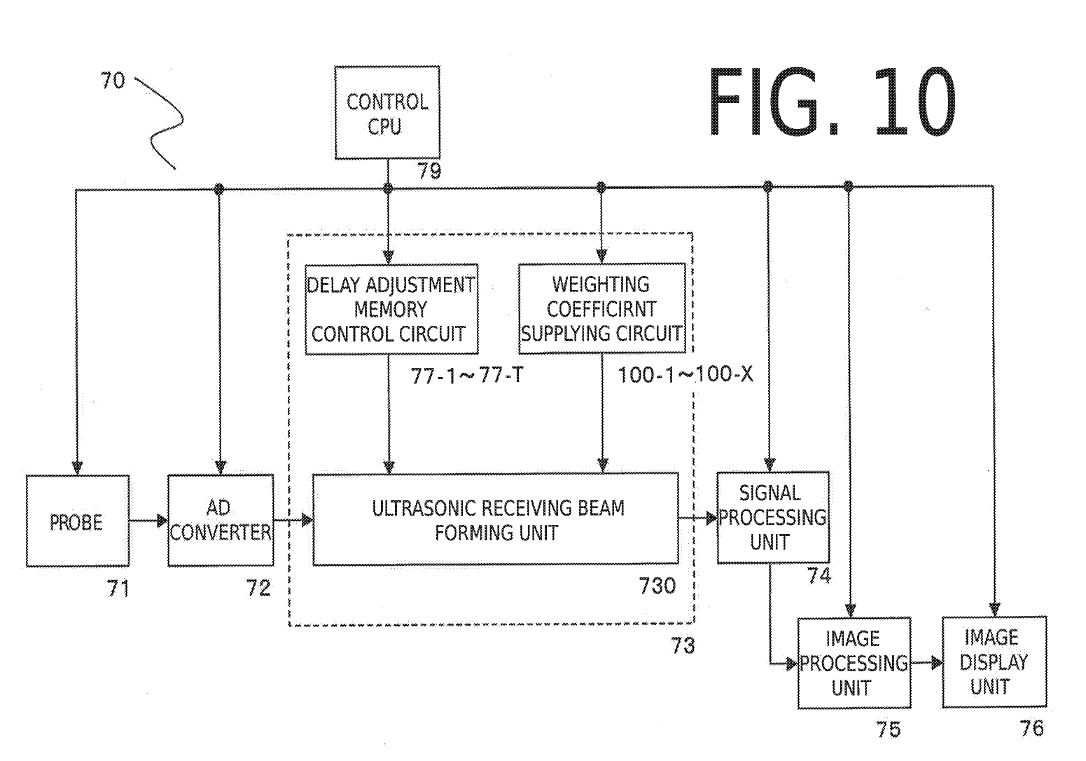

FIG. 10 is a view showing the configuration of an ultrasonic image creating system 70 using the ultrasonic receiving beam forming apparatus according to the present invention.

The ultrasonic image creating system 70 has a probe 71, an AD converter 72, an ultrasonic receiving beam forming apparatus 73, a signal processing unit 74, an image processing unit 75, an image display unit 76, and a control CPU 79. In this embodiment, the ultrasonic receiving beam forming apparatus 73 includes an ultrasonic receiving beam forming unit 730 (the ultrasonic receiving beam forming apparatus having been described in the first to the third embodiments), a delay adjustment memory control circuit 77 (77-1 to 77-T), and a weighting coefficient supplying circuit 100 (100-1 to 100-X). In this embodiment, a delay adjustment module of the ultrasonic receiving beam forming unit 730 is to receive two ultrasonic receiving signals and adjust a time lag between the signals.

The received ultrasonic signal (ultrasonic receiving data; ultrasonic receiving signal) is converted into an analog electrical signal by the probe 71, and further converted into digital signal by the AD converter 72. The digital receiving signal is subjected to the phasing addition processing by the ultrasonic receiving beam forming unit 730, and further subjected to the logarithmic compression/envelope detection processing by the signal processing unit 74. The output data of the signal processing unit 74 (the signal subjected to the logarithmic compression/envelope detection processing) is supplied to the image processing unit 75, where the data becomes image data after several processing necessary for image creation. The image display unit 76 creates an ultrasonic image from the image data created by the image processing unit 75 and displays the same. The control CPU 79 supplies data or control signal necessary for controlling each block. The delay adjustment memory control circuits 77-1 to 77-T control the respective timings of writing and reading the received signals into and from the delay adjustment memories in the ultrasonic receiving beam forming unit 730, based on the delay data (delay amount information) indicating the delay time of each ultrasonic receiving signal entered from the control CPU 79. Here, the alphabet T indicates the number of the delay adjustment memories existing in the ultrasonic receiving beam forming unit 730. The weighting coefficient supplying circuits 100-1 to 100-X supply the respective weighting coefficients to the multipliers within the ultrasonic receiving beam forming unit 730, according to the weighting coefficient data for apodization entered from the control CPU 79. Here, the alphabet X indicates the number of the multipliers for apodization existing in the ultrasonic receiving beam forming unit 730.

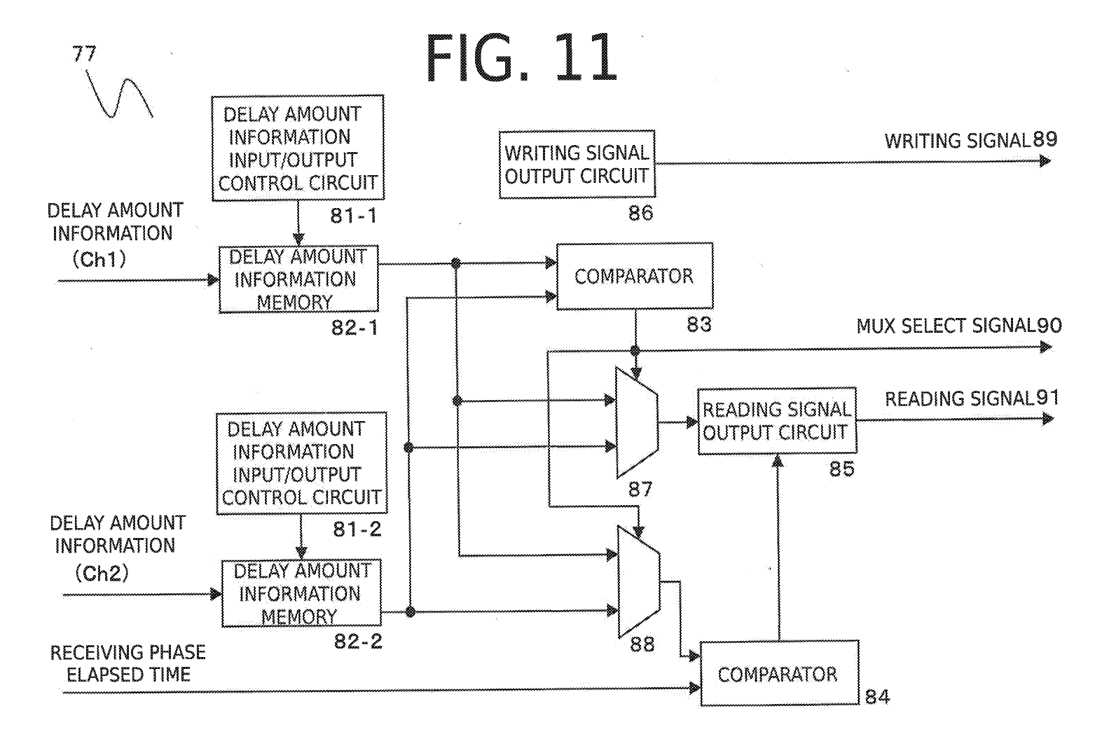

FIG. 11 is a view showing the configuration of the delay adjustment memory control circuit 77.

The delay adjustment memory control circuit 77 includes delay amount information input/output control circuits 81 (81-1 and 81-2), delay amount information memories 82(82-1 and 82-2), comparators 83 and 84, a reading signal output circuit 85, a writing signal output circuit 86, and multiplexers 87 and 88.

The delay amount information memory 82 stores the delay amount information supplied from the control CPU 79. The delay amount, information input/output control circuit 81 controls writing and reading of the delay amount information into and from the delay amount information memory 82. The writing signal output circuit 86 supplies a control signal (control data; writing signal 89) for instructing writing of the ultrasonic receiving data into the delay adjustment memory, to the delay adjustment memory. The comparator 83 makes a comparison between the respective delay times (the respective delay amount information corresponding to the Ch1 and the Ch2) of the ultrasonic data entered in the Ch1 and the Ch2 and supplies a MUX select signal 90 as the comparison result. The multiplexer 87 supplies the smaller one of the delay amount information corresponding to the Ch1 and the Ch2, according to the MUX select signal 90. The multiplexer 88 supplies the larger one of the delay amount information corresponding to the Ch1 and the Ch2, according to the MUX select signal 90. The comparator 84 compares the larger one of the delay amount information corresponding to the Ch1 and the Ch2 with the elapse time (receiving phase elapsed time) from transmission of ultrasonic wave, and supplies a reading starting trigger to the reading signal output circuit 85 when the above both values are in one accord. Upon receipt of the reading starting trigger, the reading signal output circuit 85 supplies the control signal (control data; reading signal 91) for instructing the reading of the ultrasonic receiving data from the delay adjustment memory, to the delay adjustment memory.

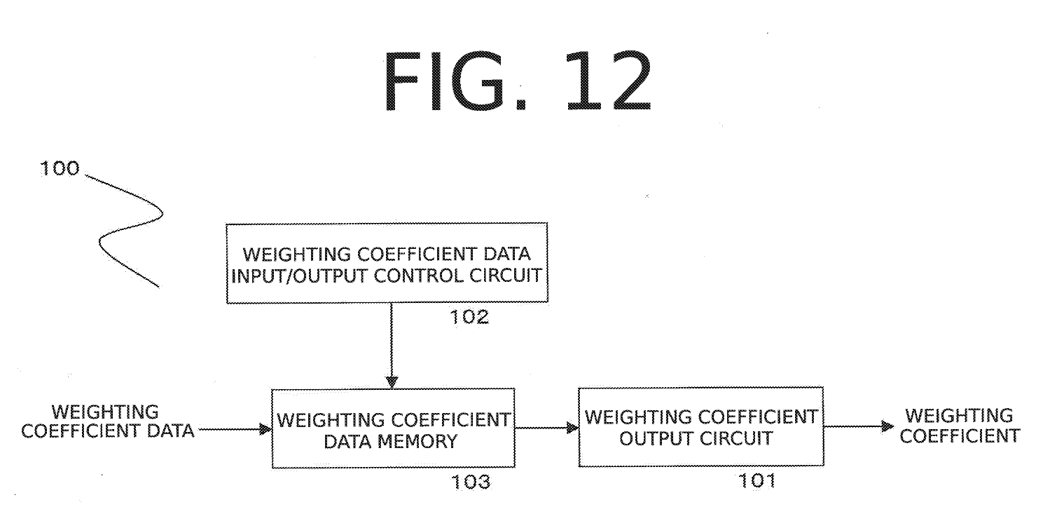

FIG. 12 is a view showing the configuration of the weighting coefficient supplying circuit 100.

The weighting coefficient supplying circuit 100 includes a weighting coefficient data input/output control circuit 102, a weighting coefficient data memory 103, and a weighting coefficient output circuit 101.

The weighting coefficient data memory 103 stores the weighting coefficient data supplied from the control CPU 79. The weighting coefficient data input/output control circuit 102 controls writing and reading of the weighting coefficient data into and from the weighting coefficient data memory 103. The weighting coefficient output circuit 101 supplies a signal necessary for apodization (data; weighting coefficient) to the multiplier, according to the weighting coefficient data supplied from the weighting coefficient data memory 103.

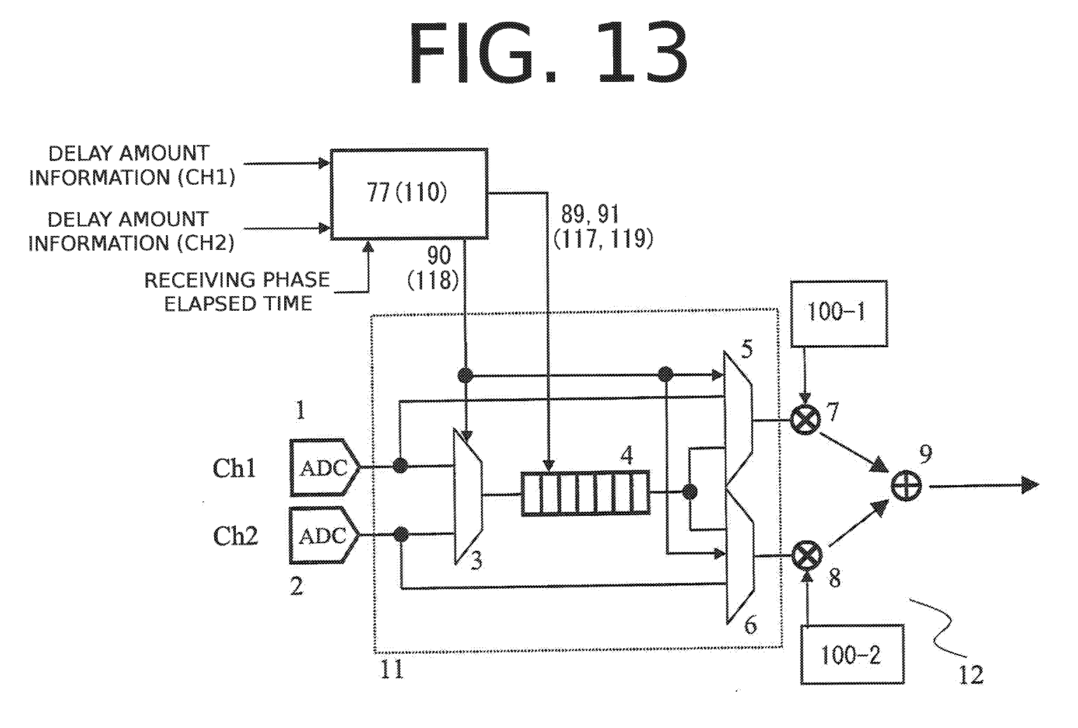

FIG. 13 is a view showing how the delay adjustment memory control circuit 77 and the weighting coefficient supplying circuits 100 are connected to the ultrasonic receiving beam forming unit (ultrasonic receiving beam forming apparatus 12). The MUX select signal 90 of the delay adjustment memory control circuit 77 is connected to the multiplexers 3, 5, and 6, hence to control the connection state of the multiplexers. The writing signal 89 and the reading signal 91 are connected to the delay adjustment memory 4, thereby to control writing and the reading of the ultrasonic receiving data into and from the delay adjustment memory 4. The weighting coefficient supplying circuits 100-1 and 100-2 are connected to the multipliers 7 and 8 respectively.

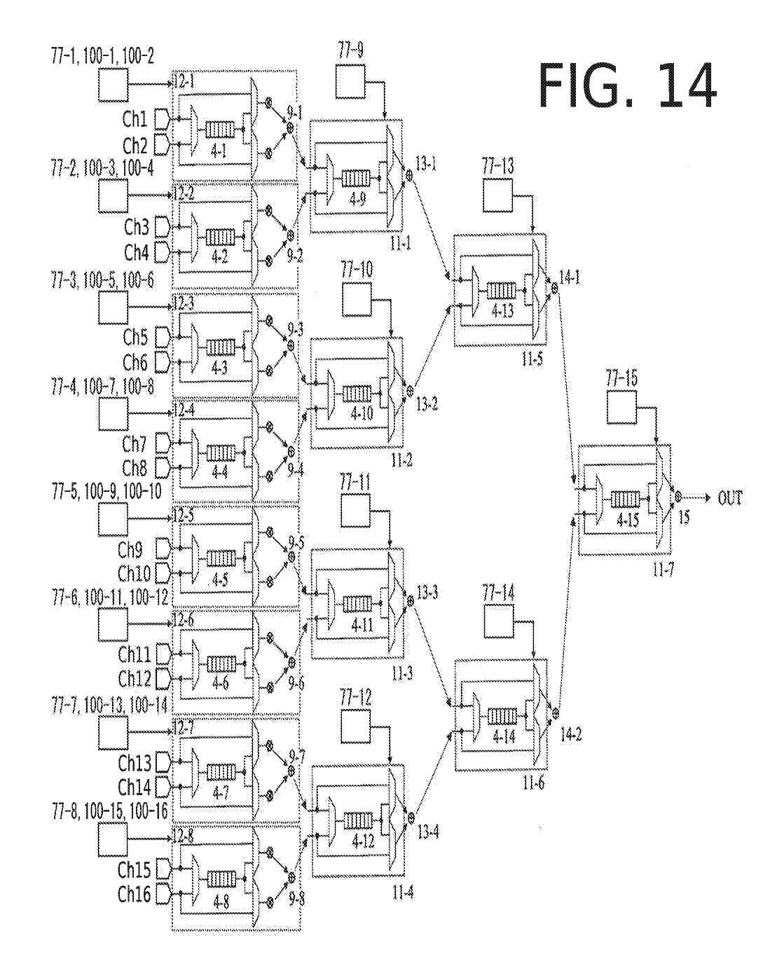

FIG. 14 is a view showing how the delay adjustment memory control circuits 77 and the weighting coefficient supplying circuits 100 are connected to the ultrasonic receiving beam forming units 730. Here, it shows the case of a 16 channel system having been described in FIG. 2 as the example of the first embodiment of the invention.

One delay adjustment memory control circuit 77 and two weighting coefficient supplying circuits 100 are arranged for the respective ultrasonic receiving beam forming apparatuses 12-1 to 12-8 (for two channels). One delay adjustment memory control circuit 77 is arranged in the respective delay adjustment modules 11-1 to 11-7. Therefore, in this case, the value of T is 15 and the value of X is 16.

The operation of the ultrasonic receiving beam forming apparatus 73 according to the fourth embodiment will be described more specifically.

With reference to FIG. 13, a case where the delay amount information is 90 in the Ch1 and 200 in the Ch2, will be described.

The delay adjustment memory control circuit 77 (delay adjustment memory control circuit 77-1) supplies the MUX select signal 90 depending on the delay amount information in the Ch1 and the Ch2. The multiplexers 3, 5, and 6 switch a connection of the receiving signal to a subsequent-stage circuit according to the MUX select signal 90. More specifically, according to the MUX select signal 90, the Chi is connected to the delay adjustment memory 4 and the Ch2 is connected to the multiplier 8. Further, the delay adjustment memory 4 is connected to the multiplier 7.

The delay adjustment memory control circuit 77 supplies the writing signal 89 to the delay adjustment memory 4. Thus, the ultrasonic receiving data in the Ch1 is written into the delay adjustment memory 4. The delay adjustment memory control circuit 77 supplies the reading signal 91 to the delay adjustment memory 4 at the timing when the ultrasonic wave reflected from a target point is received in the Ch2 (the timing when the delay information corresponding to the Ch2 corresponds with the receiving phase elapsed time). According to this, the ultrasonic receiving data of the Ch1 written in the delay adjustment memory 4 is read out. Then, the ultrasonic receiving data of the Ch1 and the Ch2 is supplied to the multipliers 7 and 8 at the same time. The multipliers 7 and 8 multiply the respective ultrasonic receiving data of the Ch1 and the Ch2 by the respective weighting coefficients supplied from the weighting coefficient supplying circuits 100-1 and 100-2. The outputs from the multipliers 7 and 8 are added together in the adder 9.

According to the above processing, the phasing addition of the Ch1 and the Ch2 is performed.

Next, the description will be made with reference to FIG. 14.

The ultrasonic receiving beam forming apparatuses 12-2 to 12-8 are controlled in the similar way as that of the ultrasonic receiving beam forming apparatus 12-1 having been described in the above; therefore, its description is omitted here (the delay adjustment memory control circuits 77-2 to 77-8 and the weighting coefficient supplying circuits 100-3 to 100-16 are used to control them). The phasing addition results (ultrasonic receiving data) of the ultrasonic receiving beam forming apparatuses 12-1 to 12-8 are passed to the delay adjustment modules 11-1 to 11-4.

The outputs of the ultrasonic receiving beam forming apparatuses 12-1 and 12-2 are phased and added in the delay adjustment module 11-1. Assume that the output time (from transmission of ultrasonic to the output of the phasing addition result) of the phasing addition result of the ultrasonic receiving beam forming apparatus 12-1 is 210 and the output time of the phasing addition result of the ultrasonic receiving beam forming apparatus 12-2 is 250. These output times may be stored in the control CPU 79 in advance or they may be calculated based on the delay amount information of the Ch1 to the Ch4.

The delay adjustment memory control circuit 77-9 compares the output time (output timing) of the phasing addition result of the ultrasonic receiving beam forming apparatus 12-1 with that of the ultrasonic receiving beam forming apparatus 12-2, and supplies the MUX select signal for connecting the phasing addition result supplied earlier, to the delay adjustment memory 4-9. According to this, the output of the ultrasonic receiving beam forming apparatus 12-1 is connected to the delay adjustment memory 4-9 and the output of the ultrasonic receiving beam forming apparatus 12-2 is connected to the adder 13-1. The delay adjustment memory 4-9 is connected to the adder 13-1.

The delay adjustment memory control circuit 77-9 supplies the writing signal 89-9 to the delay adjustment memory 4-9. According to this, the phasing addition result of the ultrasonic receiving beam forming apparatus 12-1 is written in the delay adjustment memory 4-9.

The delay adjustment memory control circuit 77-9 supplies the reading signal 91-1 to the delay adjustment memory 4-9 at the timing when the phasing addition result from the ultrasonic receiving beam forming apparatus 12-2 is supplied. According to this, the phasing addition result of the ultrasonic receiving beam forming apparatus 12-1 written in the delay adjustment memory 4-9 is read out. Then, the phasing addition result of the ultrasonic receiving beam forming apparatus 12-1 and the phasing addition result of the ultrasonic receiving beam forming apparatus 12-2 are simultaneously supplied to the adder 13-1 and added together there.

According to the above processing, the phasing addition of the Ch1 to Ch4 is performed.

The delay adjustment modules 11-2 to 11-4 are controlled in the similar way as the above mentioned delay adjustment module 11-1; therefore, the description is omitted (the delay adjustment memory control circuits 77-10 to 77-12 are used to control them). The phasing addition results of the delay adjustment modules 11-1 to 11-4 are passed to the delay adjustment modules 11-5 and 11-6.

The respective timings of writing and reading of a signal into and from the delay adjustment memories of the delay adjustment modules 11-5 and 11-6 is controlled according to the respective delay adjustment memory control circuits 77-13 and 77-14. Specifically, the timing of writing and reading of a signal into and from the delay adjustment memory of the delay adjustment module 11-5 is controlled according to the output timing of the phasing addition results from the delay adjustment modules 11-1 and 11-2. The timing of writing and reading of a signal into and from the delay adjustment memory of the delay adjustment module 11-6 is controlled according to the output timing of the phasing addition results from the delay adjustment modules 11-3 and 11-4.

Further, the timing of writing and reading a signal into and from the delay adjustment memory of the delay adjustment module 11-7 is controlled by the delay adjustment memory control circuit 77-15, according to the output timings of the phasing addition results from the delay adjustment modules 11-5 and 11-6.

According to the above-mentioned operation, the phasing addition is performed in the ultrasonic receiving beam forming apparatus 73.

Although the timing of supplying the writing signal 89 is not described in the embodiment, the writing signal 89 may be always supplied or it may be supplied based on the delay amount information (not illustrated).

### Fifth Embodiment

In this embodiment, the respective signals are switched according to a control of the circuit connecting unit (multiplexers 4, 5, and 6), based on the comparison results between the respective delay amount information of the respectively received signals and the receiving phase elapsed time. The timing of writing and reading the ultrasonic receiving signal into and from the delay adjustment memory is controlled based on the comparison results.

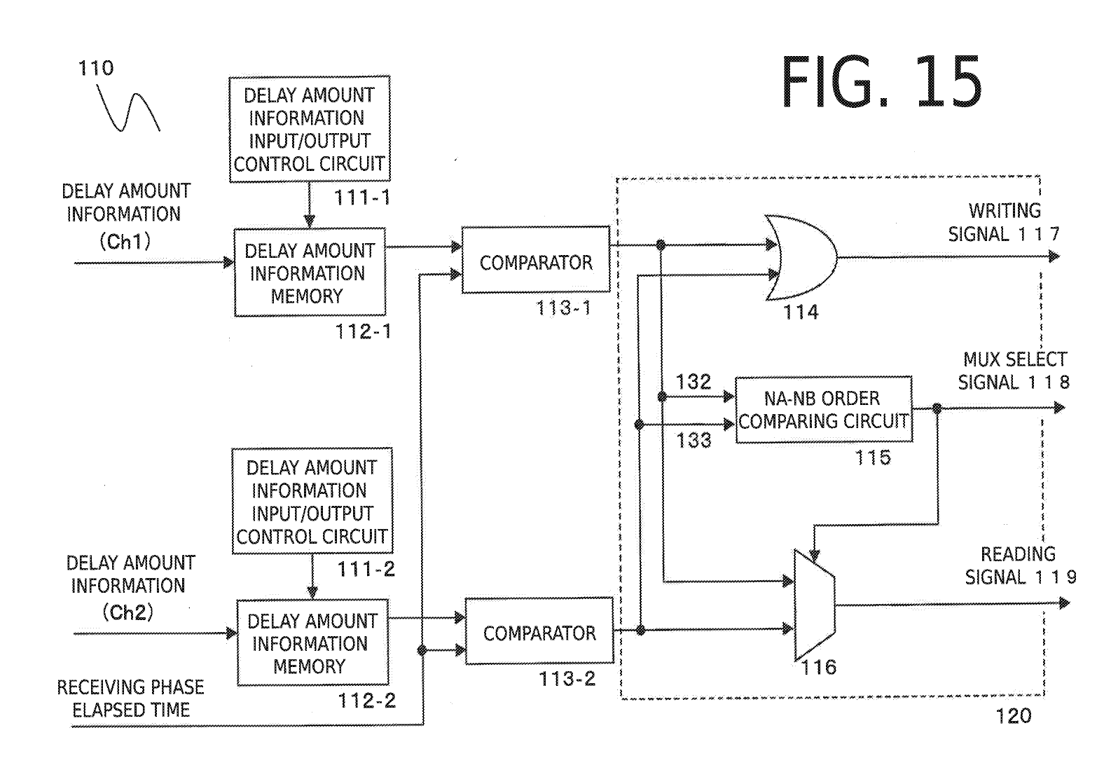

FIG. 15 is a view showing the configuration of the delay adjustment memory control circuit 110 according to the fifth embodiment of the invention.

The delay adjustment memory control circuit 110 includes delay amount information input/output control circuits 111 (111-1 and 111-2), delay amount information memories 112 (112-1 and 112-2), comparators 113 (113-1 and 113-2), an OR circuit 114, an NA-NB order comparing circuit 115, and a multiplexer 116.

The delay amount information memory 112 stores the delay amount, information supplied from the control CPU 79. The delay amount information input/output control circuit 111 controls writing and reading of the delay amount information into and from the delay amount information memory 112. The respective comparators 113-1 and 113-2 compare the respective delay amount information of the ultrasonic date entered in the Ch1 and the Ch2 with the receiving phase elapsed time and supply the respective comparison result signals 132 and 133 as the comparison result. Specifically, the initial state of the comparison result signal is “L” and the comparison result signal is switched from “L” to “H” at the timing when the receiving phase elapsed time corresponds with the delay amount information.

The NA-NB order comparing circuit 115 determines which of the comparison result signals 132 and 133 first turns into “H” and supplies the MUX select signal 118 as the determination result. Namely, the contents of the MUX select signal 118 are changed depending on which of the Ch1 and the Ch2 the ultrasonic receiving signal first arrives at.

The multiplexer 116 instructs reading of the ultrasonic receiving signal from the delay adjustment memory at the later timing of the two timings when the respective delay times of the two received ultrasonic receiving signals corresponds with the receiving phase elapsed time. Specifically, according to the MUX select signal 118, the multiplexer 116 supplies a signal which gets “H” later, of the comparison result signals 132 and 133, as the reading signal 119. It is assumed that in this embodiment, the reading processing is performed when the “H” is supplied as the reading signal 119 and that the reading processing is not performed when the “L” is supplied. According to the structure, it is possible to read the ultrasonic receiving signal from the delay adjustment memory smoothly.

The OR circuit 114 instructs writing of the ultrasonic receiving signal into the delay adjustment memory at the timing when at least one delay time of the received two ultrasonic receiving signals corresponds with the receiving phase elapsed time. Specifically, the OR circuit 114 supplies the OR result of the comparison result signals 132 and 133 as the writing signal 117. Namely, at the timing when one of the comparison result signals 132 and 133 gets “H”, the writing signal 117 becomes “H”. In this embodiment, it is assumed that when the writing signal 117 of “H” is supplied, the writing processing is performed and that when “L” is supplied, the writing processing is not performed. According to the structure, it is possible to write the ultrasonic receiving signal into the delay adjustment memory smoothly.

The connection state of the delay adjustment memory control circuit 110, the weighting coefficient supplying circuit 100, and the ultrasonic receiving beam forming apparatus 12 is similar to that of the fourth embodiment (FIG. 13) and therefore the description is omitted.

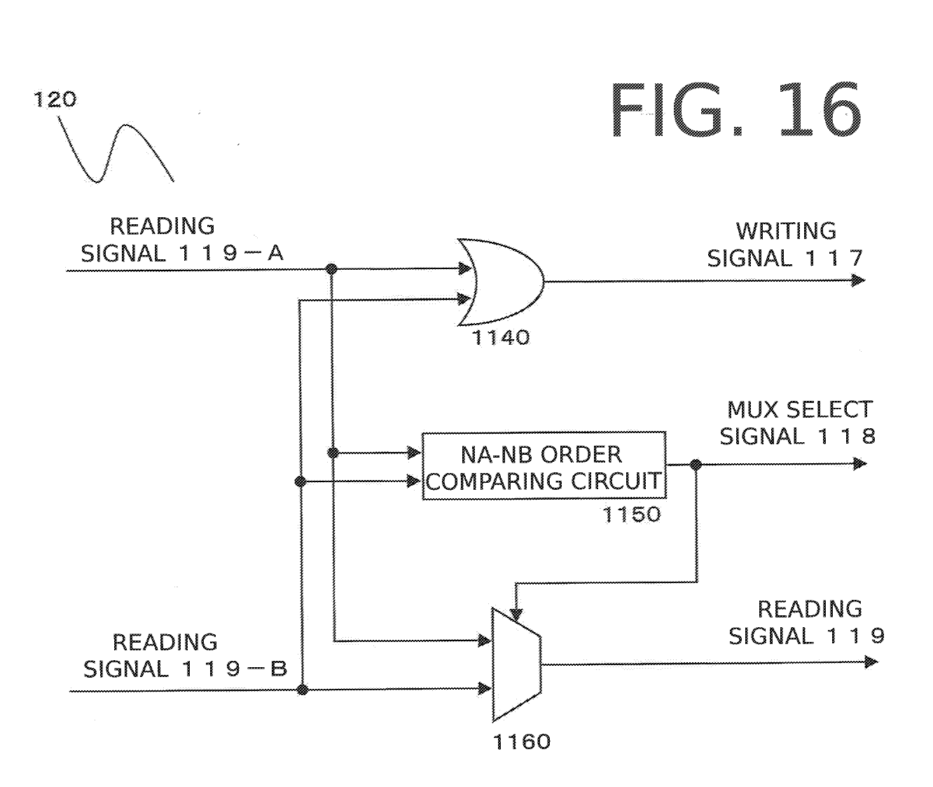

FIG. 16 is a view showing the configuration of the delay adjustment memory control circuit 120 which is arranged in the delay adjustment module 11 in the M^{th} stage (M is the integer of 2 or more) when the ultrasonic receiving beam, forming apparatus is formed in a multi-stage structure as shown in FIG. 2. The delay adjustment memory control circuit 120 is formed by a part of the delay adjustment memory control circuit 110. Namely, according to the structure of this embodiment, the configuration of the delay adjustment memory control circuits in the stages later than the second stage can be simplified. The basic operation is the same as having been described.

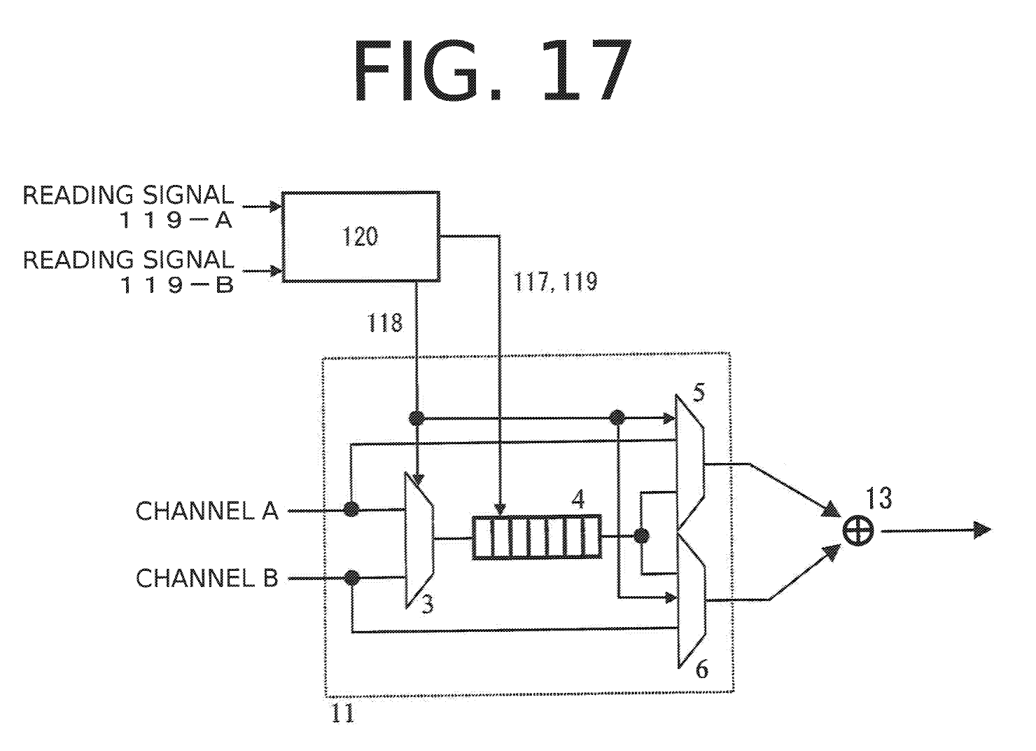

The reading signals 119-A and 119-B which are respectively supplied to the two delay adjustment modules in the (M−1)^{th} stage connected to the delay adjustment module in the M^{th} stage, are supplied to the delay adjustment memory control circuit 120. The delay adjustment memory control circuit 120 controls switching of the respective signals by the circuit connecting unit (multiplexers 4, 5, and 6) of the delay adjustment module in the M^{th} stage, according to the reading signals (FIG. 17). Further, according to these reading signals, it controls the timing of writing and reading the ultrasonic receiving signal into and from the delay adjustment memory of the delay adjustment module in the M^{th} stage (FIG. 17). FIG. 17 is a view showing how the delay adjustment memory control circuit 120 is connected to the delay adjustment module 11.

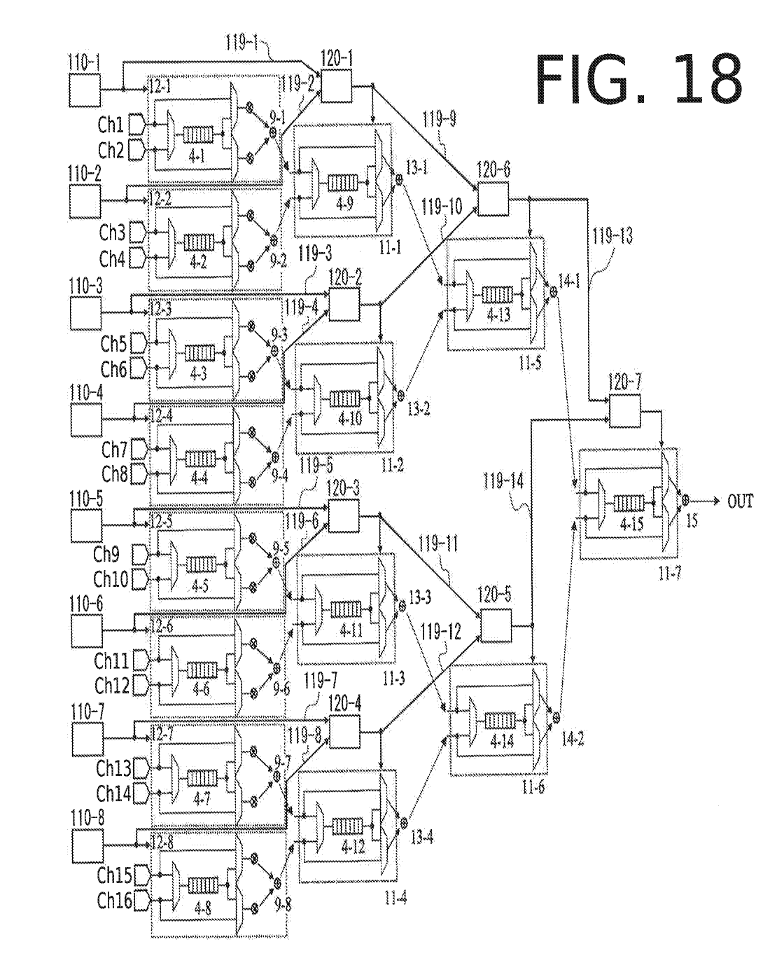

FIG. 18 is used to hereinafter describe the detailed operation of the ultrasonic receiving beam forming apparatus 73 according to the fifth embodiment. The description about the same operation as that of the fourth embodiment, is omitted.

FIG. 18 is a view showing how the delay adjustment memory control circuits 110 and 120 are connected to the ultrasonic receiving beam forming units 730 in a multi-stage structure (FIG. 18 does not illustrate the weighting coefficient supplying circuit 100). The respective delay adjustment memory control circuits 110-1 to 110-8 are arranged as for the respective ultrasonic receiving beam forming apparatuses 12-1 to 12-8 (for two channels). Further, the respective delay adjustment memory control circuits 120-1 to 120-7 are arranged as for the delay adjustment modules 11-1 to 11-7.

In the delay adjustment module 11-1, the outputs from the ultrasonic receiving beam forming apparatuses 12-1 and 12-2 are phased and added.

The reading signals 119-1 and 119-2 supplied from the delay adjustment memory control circuits 110-1 and 110-2 are supplied to the delay adjustment memory control circuit 120-1 corresponding to the delay adjustment module 11-1.

The NA-NB order comparing circuit 1150 determines which of the two delay adjustment modules in the (M−1)^{th} stage the signal “H” is first supplied to. It controls switching of the respective signals by the circuit connecting units (multiplexers 4, 5, and 6) of the delay adjustment module in the M^{th} stage, according to the determination result.

Specifically, the NA-NB order comparing circuit 1150 determines which of the reading signals 119-1 and 119-2 gets “H” first. By using the determination result, it creates and supplies the MUX select signal 118-9 (not illustrated). According to the structure, it is possible to switch the connection of the respective signals by the circuit connecting units smoothly.

For example, when the reading signal 119-1 gets “H” earlier than the reading signal 119-2, the phasing addition result (ultrasonic receiving data) of the ultrasonic receiving beam forming apparatus 12-1 is connected to the delay adjustment memory 4-9. Further, the output from the ultrasonic receiving beam forming apparatus 12-2 is connected to the adder 13-1. The delay memory 4-9 is connected to the adder 13-1.

The OR circuit 1140 instructs writing of the ultrasonic receiving signal into the delay adjustment memory of the delay adjustment module in the M^{th} stage at the timing when the reading signal “H” is supplied to at least one of the two delay adjustment, modules in the (M−1)^{th} stage.

Specifically, the OR circuit 1140 supplies the OR result of the reading signals 119-1 and 119-2 as the writing signal 117-9 (not illustrated). Namely, at the timing when one of the reading signals 119-1 and 119-2 gets “H”, the writing signal 117-9 gets “H”. According to the structure, it is possible to write the ultrasonic receiving signal into the delay adjustment memory of the delay adjustment module in the M^{th} stage smoothly.

For example, when the reading signal 119-1 gets “H” earlier than the reading signal 119-2, the phasing addition result of the ultrasonic receiving beam forming apparatus 12-1 is written in the delay adjustment memory 4-9 at the timing when the reading signal 119-1 gets “H”.

The multiplexer 1160 instructs reading of the ultrasonic receiving signal from the delay adjustment memory of the delay adjustment module in the M^{th} stage at the later one of the timings when the reading signals “H” are supplied to the two delay adjustment modules in the (M−1)^{th} stage.

Specifically, according to the MUX select signal 118-9, the multiplexer 1160 supplies that one getting “H” later, of the reading signals 119-1 and 119-2, as the reading signal 119-9. According to the structure, it is possible to read the ultrasonic receiving signal from the delay adjustment memory of the delay adjustment module in the M^{th} stage smoothly.

For example, when the reading signal 119-1 gets “H” earlier than the reading signal 119-2, the phasing addition result of the ultrasonic receiving beam forming apparatus 12-1 written in the delay adjustment memory 4-9 is read out at the timing when the reading signal 119-2 gets “H”.

The phasing addition result from the ultrasonic receiving beam forming apparatus 12-1 and the phasing addition result from the ultrasonic receiving beam forming apparatus 12-2 are simultaneously supplied to the adder 13-1 and added there.

According to the above processing, the phasing addition of the Ch1 to Ch4 is performed.

The delay adjustment modules 11-2 to 11-4 are controlled in the similar way as the above-mentioned delay adjustment module 11-1; therefore, the description is omitted (the delay adjustment memory control circuits 120-2 to 120-4 control them according to the reading signals 119-3 to 119-8). The phasing addition results of the delay adjustment modules 11-1 to 11-4 are passed to the delay adjustment modules 11-5 and 11-6.

The timings of writing and reading signals into and from the delay adjustment memories of the delay adjustment modules 11-5 and 11-6 are respectively controlled by the delay adjustment memory control circuits 120-5 and 120-6. Specifically, the timing of writing and reading a signal into and from the delay adjustment memory of the delay adjustment module 11-5 is controlled according to the reading signals 119-9 and 119-10 from the delay adjustment memory control circuits 120-1 and 120-2. The timing of writing and reading a signal into and from the delay adjustment memory of the delay adjustment module 11-6 is controlled according to the reading signals 119-11 and 119-12 from the delay adjustment memory control circuits 120-3 and 120-4.

The timing of writing and reading a signal into and from the delay adjustment memory of the delay adjustment module 11-7 is controlled by the delay adjustment memory control circuit 120-7, according to the reading signals 119-13 and 119-14 from the delay adjustment memory control circuits 120-5 and 120-6.

According to the above operation, the phasing addition is performed in the ultrasonic receiving beam forming apparatus 73.

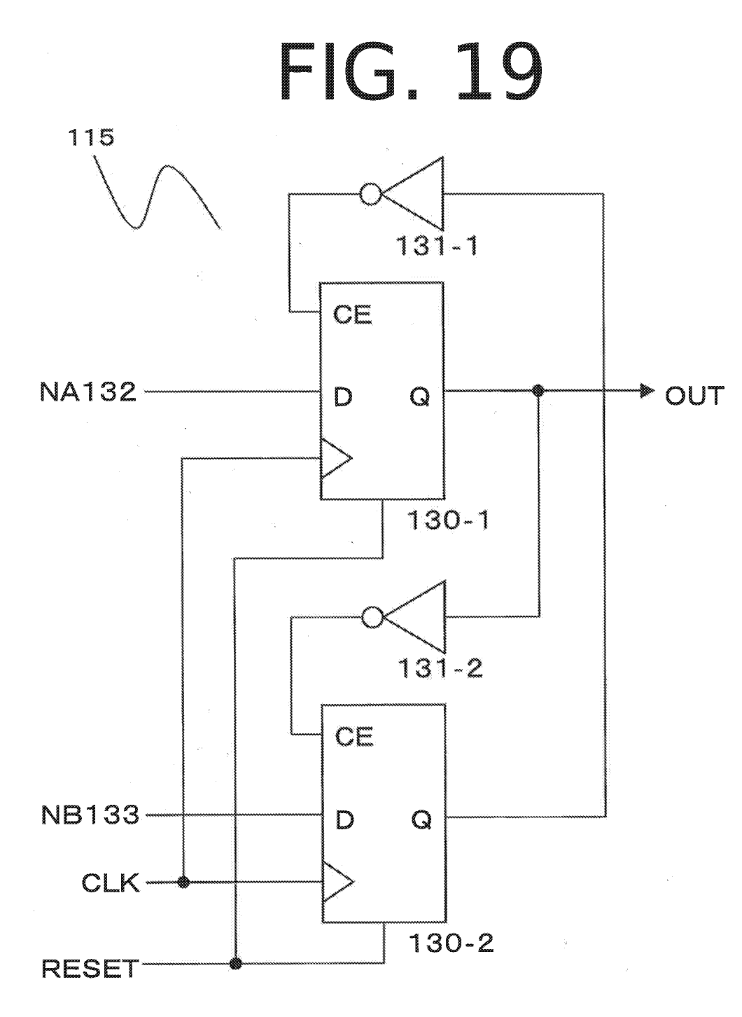

Next, the configuration of the NA-NB order comparing circuit 115 will be described using FIG. 19.

The NA-NB order comparing circuit 115 includes registers 130-1 and 130-2, and inverter circuits 131-1 and 131-2. The MUX select signal 118 of the delay adjustment memory control circuit 110 is supplied from the OUT terminal of the drawing. The configuration of the NA-NB order comparing circuit 1150 is the same as that of the NA-NB order comparing circuit 115; therefore, the description is omitted (where, as mentioned above, the NA-NB order comparing circuit 115 and the NA-NB order comparing circuit 1150 have different input signals).

The detailed operation of the NA-NB order comparing circuit 115 will be described. According to the RESET signal, the outputs of the registers 130-1 and 130-2 become the initial output “L”. After starting the ultrasonic receiving processing, when the comparison result signal 132 (NA) turns into “H” from “L” earlier than the comparison result signal 133 (NB) by one clock or more, the output of the register 130-1 becomes “H”. A little later than that, the CE (clock enable) of the register 130-2 becomes “L” according to the function of the inverter circuit 131-2. Thus, until the next RESET signal is supplied, the output of the register 130-1 is fixed at “H” and the output of the register 130-2 is fixed at “L”. The output of the register 130-1 is supplied from the OUT terminal; when the NA 132 turns into “H” from “L” earlier than the MB 133, “H” is supplied as the MUX select signal 118.

After the ultrasonic receiving phase starts, when the NB 133 turns into “H” from “L” earlier than the NA 132 by one clock or more, the output of the register 130-2 becomes “H”. A little later than that, the CE of the register 130-1 becomes “L” according to the function of the inverter circuit 131-1. Thus, the output of the register 130-2 is fixed at “H” and the output of the register 130-1 is fixed at “L” until the next RESET signal is supplied. Namely, when the NB 133 turns into “H” from “L” earlier than the NA 132, “L” is supplied as the MUX select signal 118.

Further, after the ultrasonic receiving phase starts, when the NA 132 and the NB 133 turn “H” from “L” at the same time, the outputs of the registers 130-1 and 130-2 become “H” at the same time. A little later than that, the CEs of the registers 130-1 and 130-2 becomes “L” according to the functions of the inverter circuits 131-1 and 131-2. According to this, the outputs of the registers 130-1 and 130-2 are fixed at “H” until the next RESET signal is supplied.

The configuration of the NA-NB order comparing circuit 115 is not limited to the above configuration. For example, when the NA 132 turns into “H” earlier than the NB 133, it may supply “L” as the MUX select signal 118 and when the NB 133 turns into “H” earlier than the NA 132, it may supply “H” as the MUX select signal 118.

The configuration of the delay adjustment memory control circuits 77, 110, and 120 is changeable according to the type of the delay adjustment memory 4.

The delay amount information may be supplied not from the control CPU 79 but from an external control CPU outside the ultrasonic image creating system 70 or a storing medium, or it may be calculated by an internal calculation circuit within the ultrasonic image creating system 70. It may be calculated by the internal calculation circuit within the ultrasonic image creating system 70, based on the data supplied from the control CPU 79, the external control CPU outside the ultrasonic image creating system 70, or the storing medium.

As mentioned above, the preferred embodiments of the invention have been described; however, the above embodiments have been illustrated just as an example and not to limit the scope of the invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-206831, filed on Aug. 11, 2008, and Japanese Patent Application No. 2009-155824, filed on Jun. 30, 2009, which are hereby incorporated by reference herein in their entirety.

What is claimed is:

1. A delay adjustment module for receiving two ultrasonic receiving signals and adjusting a time lag between the signals, comprising:
a storing unit for absorbing the time lag between the signals, and
a circuit connecting unit which switches a connection of the respective signals to a subsequent-stage circuit by comparing delay time of the two signals, wherein
the storing unit is shared by two signal processing channels, and
the circuit connecting unit switches a connection so that one signal having a smaller delay of the two signals is output through the storing unit and the other signal having a larger delay is output directly.

2. A delay adjustment module according to claim 1, wherein
the storing unit has a capacity capable of storing an ultrasonic receiving signal corresponding to a maximum difference of delay time between the two signals.

3. A delay adjustment module for receiving N (N is integer of 3 or more) ultrasonic receiving signals and adjusting a time lag among the signals, comprising:
a storing unit for absorbing the time lag among the signals, and
a circuit connecting unit which switches a connection of the respective signals to a subsequent-stage circuit by comparing delay time of the signals, wherein
there are N−1 pieces of the storing units, and
the circuit connecting unit switches a connection depending on the delay time of each of the signals so that the signal is output through the storing unit corresponding to the delay time thereof or output directly.

4. A delay adjustment module according to claim 3, wherein
the N−1 storing units respectively have a capacity capable of storing an ultrasonic receiving signal corresponding to a maximum difference of delay time among 2 to N signals.

5. A delay adjustment, module according to claim 1, wherein
the storing unit is any one of a FIFO memory, a random access memory of single port, and a random access memory of dual port.

6. A delay adjustment module according to claim 3, wherein
the storing unit is any one of a FIFO memory, a random access memory of single port, and a random access memory of dual port.

7. An ultrasonic receiving beam forming apparatus comprising:
the delay adjustment module according to claim 1, and
an adding unit which adds the ultrasonic receiving signals after the time lag between the signals is adjusted by the delay adjustment module.

8. An ultrasonic receiving beam forming apparatus comprising:
the delay adjustment module according to claim 3, and
an adding unit which adds the ultrasonic receiving signals after the time lag between the signals is adjusted by the delay adjustment module.

9. An ultrasonic receiving beam forming apparatus according to claim 7, comprising
a multiplying unit which assigns weight to the output from the delay adjustment module.

10. An ultrasonic receiving beam forming apparatus according to claim 1, wherein
the delay adjustment modules and the adding units are formed in a multi-stage structure.

11. An ultrasonic receiving beam forming apparatus according to claim 10, further comprising
a multiplying unit which assigns weight to the output from the delay adjustment module in a first stage.

12. An ultrasonic receiving beam forming apparatus according to claim 7, wherein
multiple beams are formed by processing at an operation frequency several times more than a sampling frequency.

13. An ultrasonic receiving beam forming apparatus according to claim 7, further comprising
a control unit which controls a timing of writing and reading of the ultrasonic receiving signal into and from the storing unit.

14. An ultrasonic receiving beam forming apparatus according to claim 13, wherein
the delay adjustment module is a delay adjustment module for receiving two ultrasonic receiving signals and adjusting a time lag between the signals, and
the control unit controls the circuit connecting unit to switch a connection of the respective signals, based on comparison result of the delay times of the signals and an elapsed time from transmission of an ultrasonic, and controls the timing of writing and reading of the ultrasonic receiving signal into and from the storing unit.

15. An ultrasonic receiving beam, forming apparatus according to claim 14, wherein
the control unit instructs writing of the ultrasonic receiving signal into the storing unit at a timing when at least one delay time of the two ultrasonic receiving signals corresponds with the elapsed time.

16. An ultrasonic receiving beam forming apparatus according to claim 14, wherein
the control unit instructs reading of the ultrasonic receiving signal from the storing unit at a later timing, of the two timings when the respective delay times of the two ultrasonic receiving signals correspond with the elapsed time.

17. An ultrasonic receiving beam forming apparatus according to claim 14, wherein
the delay adjustment modules and the adding units are formed in a multi-stage structure, and
the control unit controls the circuit connecting unit of the delay adjustment module in an M^{th} (M is integer of 2 or more) stage to switch a connection of the respective signals, based on a control signal for instructing reading of the ultrasonic receiving signals which are respectively supplied to the two delay adjustment modules in (M−1)^{th} stage connected to the delay adjustment module in the M^{th} stage, and controls a timing of writing and reading of the ultrasonic receiving signal into and from the storing unit of the delay adjustment module in the M^{th} stage.

18. An ultrasonic receiving beam forming apparatus according to claim 17, wherein
the control unit instructs writing of the ultrasonic receiving signal into the storing unit of the delay adjustment module in the M^{th} stage at a timing when the control signal is supplied to at least one of the two delay adjustment modules in the (M−1)^{th} stage.

19. An ultrasonic receiving beam forming apparatus according to claim 17, wherein
the control unit determines which of the two delay adjustment modules in the (M−1)^{th} stage the control signal is first supplied to, and controls the circuit connecting unit of the delay adjustment module in the M^{th} stage to switch a connection of the respective signals, using the determination result.

20. An ultrasonic receiving beam forming apparatus according to claim 17, wherein
the control unit instructs reading of the ultrasonic receiving signal from the storing unit of the delay adjustment module in the M^{th} stage at a later timing, of the two timings when the control signals are supplied respectively to the two delay adjustment modules in the (M−1)^{th} stage.
